(19) United States
(12) Patent Application Publication
(10) Pub. No.: US 2023/0321117 A1
(43) Pub. Date: Oct. 12, 2023

(54) USE OF GANAXOLONE IN TREATING AN EPILEPSY DISORDER

(71) Applicant: MARINUS PHARMACEUTICALS, INC., Radnor, PA (US)

(72) Inventors: Alex Aimetti, Radnor, PA (US); Joseph HULIHAN, Radnor, PA (US)

(21) Appl. No.: 18/328,996

(22) Filed: Jun. 5, 2023

Related U.S. Application Data
(63) Continuation of application No. PCT/US21/61937, filed on Dec. 6, 2021.
(60) Provisional application No. 63/122,378, filed on Dec. 7, 2020.

(51) Int. Cl. A61K 31/57; A61K 9/48; A61K 9/00; A61P 25/08

(52) U.S. Cl. CPC A61K 31/57; A61K 9/4808; A61B 5/291; A61P 25/08; A61K 9/0053

(57) ABSTRACT

The disclosure to methods for treating seizure or an epilepsy disorder comprising administering to a subject in need thereof a therapeutically effective amount of ganaxolone or a pharmaceutically acceptable salt thereof.

### 1. BACKGROUND

Epilepsy is a chronic neurological disorder characterized by repeated seizures (>24 hours apart); by one seizure with a strong potential for recurrence (at least 60%). Chen et al., (2018), JAMA Neurol., 75(3):279-286. It affects people of all ages and results in social, behavioral, health, and economic consequences to the patients and their families. Id. It is estimated that more than 50 million people worldwide are effected. Id.

Antiepileptic drugs are the mainstay of treatment and suppress seizure occurrence without rectifying the underlying neuropathological process. Id. As a result, people with epilepsy often require life-long antiepileptic drug treatment. Additional therapeutic options include respective surgery, brain stimulation, and dietary therapy. In everyday practice, the choice of initial antiepileptic drugs on diagnosis currently depends mostly on syndromic classification, the treatment choice being framed by whether the epilepsy is deemed to be genetic generalized or focal in onset. Scheffer et al., (2017), Epilepsia, 58(4):512-21. This crude separation of the epilepsies depends on the clinical and electroencephalography features present at the time of diagnosis. Id. Moreover, despite new drugs for epilepsy, the rate of seizure freedom has remained largely unchanged across the decades.

Accordingly, there is a significant unmet need for safe and effective therapies for treating epilepsy disorders.

### 2. SUMMARY

This disclosure relates to methods for treating an epilepsy disorder.

Ganaxolone has been suggested and studied as a therapy for particular seizure and epilepsy-related disorders (e.g., status epilepticus and CDLK5 deficiency disorder). However, the results of such investigations have been variable, and conventional wisdom is that ganaxolone generally has to be administered at a very high dose to be effective. For example, in a clinical trial in adults with focal seizures, ganaxolone was administered orally at a daily dose of 1800 mg (administered as 900 mg twice a day), yet the clinical endpoint was not achieved.

The inventors have now surprisingly discovered that administering ganaxolone at a lower dose but with more frequent administration can result in effective treatment of seizure and epilepsy disorders. Without wishing to be bound by any particular theory or mechanism, it is believed that more frequent administration of ganaxolone (e.g., three times a day or more) but at the same or lower daily dose as used previously in clinical trials, improves drug exposure by maintaining a ganaxolone serum level (e.g., trough levels) above a threshold level for longer periods of time resulting in effective treatment. As described and exemplified herein, administering ganaxolone three times daily (or more) with a total daily dose of no more than 1800 mg, or no more than 1700 mg, or no more than 1600 mg, or no more than 1500 mg, or no more than 63 mg/kg/day of ganaxolone can produce a plasma concentration of ganaxolone of at least about 100 ng/ml for approximately 70% or greater for a 24 hour-day and provide effective seizure reduction.

Also, less ganaxolone in comparison to the amounts that were administered in prior clinical trials (e.g., total daily dose) is typically required to achieve the increased trough concentration of ganaxolone using three times per day (or more frequent) administration, which is also beneficial for the subject under treatment. In fact, trough ganaxolone levels with twice a day dosing typically remained below 100 ng/ml over the 24 hour treatment period.

Accordingly, this disclosure relates to methods for treating an epilepsy disorder. The methods disclosed herein comprise administering to a subject in need thereof a therapeutically effective amount of ganaxolone or a pharmaceutically acceptable salt thereof. Ganaxolone is preferably administered in an amount that will provide a trough ganaxolone level (e.g., a ganaxolone plasma concentration) of about 100 ng/ml or greater for approximately 70% or greater for a 24 hour-day (e.g., each day during the treatment period).

To achieve a ganaxolone plasma concentration of about 100 ng/ml or greater for approximately 70% or greater over a 24 hour, ganaxolone can be administered three times a day at a total daily dose of no more than 1800 mg, or more than 1700 mg, or no more than 1600 mg, or no more than 1500 mg or less, or no more than 63 mg/kg/day. In subjects' that weigh less than 40 kg ganaxolone can be administered three times a day at a maximum amount of 63 mg/kg/day. Typically, up to about 1,500 mg per day of ganaxolone can produce a plasma concentration of about 100 ng/ml or greater for approximately 70% or greater over a 24 day when administered three times a day. Ganaxolone can be administered (e.g., orally) at a dose of about 500 mg three times a day. A skilled clinician will understand that the amount of ganaxolone administered three times a day (e.g., orally) can be adjusted to achieve the desired ganaxolone trough level so long as the total amount does not exceed the maximum daily dose of ganaxolone.

Preferably ganaxolone is administered orally (e.g., as an oral suspension or an oral capsule). Without being bound by theory, the inventors believe that three time dosing results in improved antiseizure activity (i.e., reduces seizure frequency) because of increased plasma ganaxolone exposure with trough levels that remain about 100 ng/mL for at least about 70% of a 24 hour day. This is contrary to prior treatment protocols that involved administering ganaxolone at high drug dose (e.g. a per day dose) to achieve therapeutic efficacy. For example, administering ganaxolone at a higher dose two times a day.

Administering ganaxolone at an amount sufficient to achieve a ganaxolone plasma concentration of at least about 100 ng/ml or greater for approximately about 70% or greater over a 24 day reduces the frequency of seizure in the subject relative to baseline. Typically, a reduction in seizure frequency of at least about 20% or greater relative to baseline seizure frequency can be achieved. During treatment, the plasma concentration of ganaxolone in the subject can be monitored, and/or the subject can be monitored for seizure activity using EEG. If the subject appears to show signs of seizure (e.g., seizure recurrence), the amount of ganaxolone administered can be adjusted accordingly.

The methods disclosed can be used to treat any desired form of seizure or epilepsy disorder. For example, the method can be used to treat focal seizures (e.g. temporal lobe seizures, frontal lobe seizures, occipital lobe seizures, or parietal lobe seizures), generalized seizures (e.g., absence seizures, myoclonic seizures, generalized tonic-clonic seizures), progressive myoclonic epilepsy, reflex epilepsy, Landau-Kleffner Syndrome, Ohtahara syndrome, Rasmussen's syndrome, infantile spasms (or West syndrome), Lennox-Gastaut syndrome (LGS), Rett syndrome, Dravet syndrome, Doose syndrome, CDKL5 disorder, intractable childhood epilepsy (ICE), childhood absence epilepsy (CAE), juvenile myoclonic epilepsy (JME), essential tremor, acute repetitive seizures, benign rolandic epilepsy, status epilepticus, refractory status epilepticus, super-refractory status epilepticus, PCDH19 pediatric epilepsy, increased seizure activity or breakthrough seizures (increased seizure activity; also called serial or cluster seizures), infantile spasms, and the like.

### 1. BRIEF DESCRIPTION OF DRAWINGS

FIG. 1 is a bivariate correlation of percent change in major motor seizures and mean plasma ganaxolone concentration. Analysis uses the same population (N=38) as used for linear regression. Logₑ percent change in major motor seizure frequency was calculated as Logₑ (percent change+100). Axes with equivalent ganaxolone level and equivalent percent change in major motor seizure frequency are displayed to approximate non-transformed values.

FIG. 2 is a graph comparing the median percent reduction in major motor seizures according to tertiles based on mean plasma ganaxolone concentrations.

FIG. 3 Modeled PK curves for TID vs. BID dosing of oral ganaxolone at 1,800 mg/day.

FIG. 4 is a linear regression of percentage change in major motor seizures with plasma ganaxolone concentration as an independent variable.

### 2. DETAILED DESCRIPTION

Effectively treating seizure and/or epilepsy disorders has been challenging and conventional treatment protocols are not effective in many patients. This disclosure relates to the discovery that administering ganaxolone at the same or lower daily dose than has been used previously in clinical trials, but with more frequent administration can provide an effective therapy for treating seizure and/or epilepsy disorders.

The methods described herein comprise administering a therapeutically effective amount of ganaxolone to a subject in need thereof that is sufficient to achieve a plasma concentration of ganaxolone of 100 ng/ml or higher for approximately 70% or more of 24 hour day (e.g., each day of a treatment period). This can be achieved by administering ganaxolone at least three times a day. Three times a day is preferable, although in some instances it may be appropriate to administer ganaxolone more than three times a day, for example, if needed or desired to achieve the desired trough concentration of ganaxolone for at least about 70% or more of a 24 hour day. A plasma concentration of at least about 100 ng/ml or more for approximately 70% or greater of a 24 hour day result in improved seizure reduction and/or suppression of seizure. For example, a seizure reduction of at least 20% or greater relative to baseline seizure frequency can be achieved. The same or often a lower total daily dose of ganaxolone than has been used in prior clinical studies can typically be used to treat a subject in need thereof, when administered at least three times per day in accordance with this disclosure. For example a lower daily dose of ganaxolone administered using an administration regimen that involves three separate administrations (e.g., preferably oral) can be used to a subject with seizure and/or epilepsy disorders. Again, without being bound by any particular theory, this is believed to provide improved clinical response to treatment because the administration regimen achieve a desired trough concentration of ganaxolone for the desired time in the subject (e.g., at least about 100 ng/ml or more for approximately 70% or greater of a 24 hour day). Typically, a maximum daily dose of about 1,800 mg, about 1,700 mg, about 1,600 mg, preferably about 1,500 mg, less than 1,500 mg or about 63 mg/kg/day of ganaxolone may be administered in accordance with this disclosure. The maximum daily dose of ganaxolone is administered at the same or varying doses across at least three intervals in a 24 hour-day.

The methods can be used to treat any form of seizure and/or epilepsy disorder. Suitable seizure and/or epilepsy disorders that can be treated with ganaxolone as disclosed herein include, but are not limited to, focal seizures (e.g. temporal lobe seizures, frontal lobe seizures, occipital lobe seizures, or parietal lobe seizures), generalized seizures (e.g., absence seizures, myoclonic seizures, generalized tonic-clonic seizures), progressive myoclonic epilepsy, reflex epilepsy, Landau-Kleffner Syndrome, Ohtahara syndrome, Rasmussen's syndrome, infantile spasms (or West syndrome), Lennox-Gastaut syndrome (LGS), Rett syndrome, Dravet syndrome, Doose syndrome, CDKL5 disorder, intractable childhood epilepsy (ICE), childhood absence epilepsy (CAE), juvenile myoclonic epilepsy (JME), essential tremor, acute repetitive seizures, benign rolandic epilepsy, status epilepticus, refractory status epilepticus, super-refractory status epilepticus, PCDH19 pediatric epilepsy, increased seizure activity or breakthrough seizures (increased seizure activity; also called serial or cluster seizures), and infantile spasms.

### A. Methods of Treating

The disclosure relates to methods for treating seizure and/or epilepsy disorders. As described herein, treatment according to the methods provides a reduced seizure burden (e.g., frequency of seizures) and/or suppression of seizure.

The method comprises administering to a subject in need thereof a therapeutically effective amount of ganaxolone to achieve a plasma concentration of ganaxolone of 100 ng/ml or higher for approximately 70% or more of a 24 hour-day. This is typically achieved by administering ganaxolone (e.g., orally) at least three times a day. For instance, ganaxolone can be administered three times a day, four times a day, five times a day, six times a day, seven times a day, eight times a day or more. Three times a day is preferable, although if needed or desired ganaxolone can be administered more than three times a day to achieve the desired trough concentration of ganaxolone.

Ganaxolone administered in accordance with the methods disclosed herein can be administered at the same or a lower daily dose that have been used in clinical trials, and can increase drug exposure by maintaining a ganaxolone serum concentration of at least about 100 ng/ml, e.g. for at least about 70% or more of a 24 hour day. A total daily dose of about 1800 mg, about 1700 mg, about 1600 mg, about 1500 mg or 63 mg/kg/day of ganaxolone can be administered, provided that the total daily dose is administered in three or more separate administrations (which preferably each contain the same amount of ganaxolone) to produce a serum ganaxolone concentration of at least about 100 ng/ml for at least 70% or more for a 24-hour day. Typically, a total daily dose of 1500 mg per day of ganaxolone can produce a plasma concentration of at least about 100 ng/ml or greater for approximately 70% or more over a 24 hour day when administered three times a day during the day.

For example, when a total daily dose of 1500 mg of ganaxolone is administered, a dose of about 500 mg three times a day can be administered. For example, when a total daily dose of 1800 mg of ganaxolone is administered, a dose of 600 mg three times a day can be administered. The maximum daily dose of ganaxolone is administered at the same or varying doses across at least three intervals in a 24 hour period. A skilled clinician will understand that the amount of ganaxolone administered at least three times a day can be adjusted to achieve the desired ganaxolone trough level so long as the total amount does not exceed the maximum daily dose of ganaxolone.

While a plasma concentration of at least about 100 ng/ml is preferable, there can be some variability based on, for example, differences in subjects' weight, metabolism, age, duration of seizure and severity of seizure.

Ganaxolone can be administered orally (e.g., as an oral suspension or an oral capsule) or an intravenous formulation. Preferably, ganaxolone is administered orally. Oral administration can include, but not limited to, oral suspension formulations and oral capsules.

A plasma concentration of at least about 100 ng/ml or higher for approximately 70% or greater of a 24 hour-day can result in improved seizure reduction and/or suppression of seizure. For example, a seizure reduction of at least 20% or greater relative to baseline seizure frequency can be achieved. For example, a seizure reduction of at least 35% or greater relative to baseline frequency can be achieved. Seizure burden and/or frequency can be using EEG.

The methods disclosed herein are suitable to treat any form of epilepsy disorder such as, focal seizures (e.g. temporal lobe seizures, frontal lobe seizures, occipital lobe seizures, or parietal lobe seizures), generalized seizures (e.g., absence seizures, myoclonic seizures, generalized tonic-clonic seizures), progressive myoclonic epilepsy, reflex epilepsy, Landau-Kleffner Syndrome, Ohtahara syndrome, Rasmussen's syndrome, infantile spasms (or West syndrome), Lennox-Gastaut syndrome (LGS), Rett syndrome, Dravet syndrome, Doose syndrome, CDKL5 disorder, intractable childhood epilepsy (ICE), childhood absence epilepsy (CAE), juvenile myoclonic epilepsy (JME), essential tremor, acute repetitive seizures, benign rolandic epilepsy, status epilepticus, refractory status epilepticus, super-refractory status epilepticus, PCDH19 pediatric epilepsy, increased seizure activity or breakthrough seizures (increased seizure activity; also called serial or cluster seizures), infantile spasms, and the like.

### B. Formulations

Any desired formulation that comprise a therapeutically effective amount of ganaxolone can be administered according to the methods disclosed herein.

The formulation is preferably an oral formulation of ganaxolone. In certain embodiments, a liquid formulation as described and prepared in U.S. Pat. No. 8,022,054, entitled “Liquid Ganaxolone Formulations and Methods for the Making and Use Thereof”, hereby incorporated by reference in its entirety, is used. The oral liquid (e.g., suspension) formulation of ganaxolone may be prepared using any suitable methods.

As described in U.S. Pat. No. 8,022,054, the liquid formulation may be an aqueous dispersion of stabilized particles comprising ganaxolone, a hydrophilic polymer, a wetting agent, and an effective amount of a complexing agent that stabilizes particle growth after an initial particle growth and endpoint is reached, the complexing agent selected from the group of small organic molecules having a molecular weight less than 550 and containing a moiety selected from the group consisting of a phenol moiety, an aromatic ester moiety and an aromatic acid moiety, wherein the stabilized particles have a volume weighted median diameter (D50) of the particles from about 50 nm to about 500 nm, the complexing agent being present in an amount from about 0.05% to about 5%, w/w based on the weight of particles, the particles dispersed in an aqueous solution which further contains at least two preservatives in an amount sufficient to inhibit microbial growth. The hydrophilic polymer may be in an amount from about 3% to about 50%, w/w, based on the weight of the solid particles. The wetting agent may be an amount from about 0.01% to about 10%, w/w, based on the weight of the solid particles. Ganaxolone may be in an amount from about 10% to about 80% (and in certain embodiments form about 50% to about 80%) based on the weight of the stabilized particles. The stabilized particles may exhibit an increase in volume weighted median diameter (D50) of not more than about 150% when the particles are dispersed in simulated gastric fluid (SGF) or simulated intestinal fluid (SIF) at a concentration of 0.5 to 1 mg ganaxolone/mL and placed in a heated bath at 36° to 38° C. for 1 hour as compared to the D50 of the stabilized particles when the particles are dispersed in distilled water under the same conditions, wherein the volume weighted median diameter (D50) of the stabilized particles dispersed in SGF or SIF is less than about 750 nm. The stabilized particles may exhibit an increase in volume weighted median diameter (D50) of not more than about 150% when the formulation is dispersed in 15 mL of SGF or SIF at a concentration of 0.5 to 1 mg ganaxolone/mL as compared to the D50 of the stabilized particles when the particles are dispersed in distilled water under the same conditions, wherein the volume weighted median diameter (D50) of the stabilized particles dispersed in SGF or SIF is less than about 750 nm.

The complexing agent can be any molecule with a lipophilic core and hydrophilic outer shell capable of solubilizing ganaxolone. In certain embodiments, complexing agent can be a substance containing a phenol moiety, an aromatic ester moiety or an aromatic acid moiety. In certain embodiments, complexing agents are selected from the group consisting of parabens, organic acids, carboxylic acids, aromatic acids, aromatic esters, acid salts of amino acids, methyl anthranilate, sodium metabisulphite, ascorbic acid and its derivatives, malic acid, isoascorbic acid, citric acid, tartaric acid, sodium sulphite, sodium bisulphate, tocopherol, water- and fat-soluble derivatives of tocopherol, sulphites, bisulphites and hydrogen sulphites, para-aminobenzoic acid and esters, 2,6-di-t-butyl-alpha-dimethylamino-p-cresol, t-butylhydroquinone, di-t-amylhydroquinone, di-t-butylhydroquinone, butylhydroxytoluene (BHT), butylhydroxyanisole (BHA), pyrocatechol, pyrogallol, propyl/gallate, nordihydroguaiaretic acid, phosphoric acids, sorbic and benzoic acids, esters, ascorbyl palmitate, derivatives and isomeric compounds thereof, pharmaceutically acceptable salts thereof, and mixtures thereof. In certain embodiments, the complexing agent is selected from the group consisting of a paraben, benzoic acid, phenol, sodium benzoate, methyl anthranilate, and the like. The hydrophilic polymer may be a cellulosic polymer, a vinyl polymer and mixtures thereof. The cellulosic polymer may be a cellulose ether, e.g., hydroxypropymethylcellulose. The vinyl polymer may be polyvinyl alcohol, e.g., vinyl pyrrolidone/vinyl acetate copolymer (S630). The wetting agent may be sodium lauryl sulfate, a pharmaceutically acceptable salt of docusate, and mixtures thereof. The aqueous dispersion may further comprise a sweetener, e.g., sucralose. In certain embodiments, the preservative is selected from the group consisting of potassium sorbate, methylparaben, propylparaben, benzoic acid, butylparaben, ethyl alcohol, benzyl alcohol, phenol, benzalkonium chloride, and mixtures of any of the foregoing.

In some embodiments, liquid ganaxolone formulations comprising the ganaxolone particles described herein and at least one dispersing agent or suspending agent for oral administration to a subject are used. The ganaxolone formulation may be a powder and/or granules for suspension, and upon admixture with water, a substantially uniform suspension is obtained. As described herein, the aqueous dispersion can comprise amorphous and non-amorphous ganaxolone particles of consisting of multiple effective particle sizes such that ganaxolone particles having a smaller effective particle size are absorbed more quickly and ganaxolone particles having a larger effective particle size are absorbed more slowly. In certain embodiments, the aqueous dispersion or suspension used in the methods disclosed herein is an immediate release formulation. In another embodiment, an aqueous dispersion comprising amorphous ganaxolone particles is formulated such that about 50% of the ganaxolone particles are absorbed within about 3 hours after administration and about 90% of the ganaxolone particles are absorbed within about 10 hours after administration. In other embodiments, addition of a complexing agent to the aqueous dispersion results in a larger span of ganaxolone containing particles to extend the drug absorption phase such that 50-80% of the particles are absorbed in the first 3 hours and about 90% are absorbed by about 10 hours.

A suspension is “substantially uniform” when it is mostly homogenous, that is, when the suspension is composed of approximately the same concentration of ganaxolone at any point throughout the suspension. Preferred embodiments are those that provide concentrations essentially the same (within 15%) when measured at various points in a ganaxolone aqueous oral formulation after shaking. Especially preferred are aqueous suspensions and dispersions, which maintain homogeneity (up to 15% variation) when measured 2 hours after shaking. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

In some embodiments, ganaxolone formulations are powders for aqueous dispersion and comprise stable ganaxolone particles having an effective particle size by weight of less than 500 nm formulated with ganaxolone particles having an effective particle size by weight of greater than 500 nm. In such embodiments, the formulations have a particle size distribution wherein about 10% to about 100% of the ganaxolone particles by weight are between about 75 nm and about 500 nm, about 0% to about 90% of the ganaxolone particles by weight are between about 150 nm and about 400 nm, and about 0% to about 30% of the ganaxolone particles by weight are greater than about 600 nm. The ganaxolone particles describe herein can be amorphous, semi-amorphous, crystalline, semi-crystalline, or mixture thereof.

In one embodiment, the aqueous suspensions or dispersions described herein comprise ganaxolone particles or ganaxolone complex at a concentration of about 20 mg/ml to about 150 mg/ml of suspension. In another embodiment, the aqueous oral dispersions described herein comprise ganaxolone particles or ganaxolone complex particles at a concentration of about 25 mg/ml to about 75 mg/ml of solution. In yet another embodiment, the aqueous oral dispersions described herein comprise ganaxolone particles or ganaxolone complex at a concentration of about 50 mg/ml of suspension. The aqueous dispersions described herein are especially beneficial for the administration of ganaxolone to infants (less than 2 years old), children under 10 years of age and any patient group that is unable to swallow or ingest solid oral dosage forms.

Liquid ganaxolone formulation for oral administration can be aqueous suspensions selected from the group including, but not limited to, pharmaceutically acceptable aqueous oral dispersions, emulsions, solutions, and syrups. See, e.g., Singh et al., Encyclopedia of Pharmaceutical Technology, 2nd Ed., pp. 754-757 (2002). In addition to ganaxolone particles, the liquid dosage forms may comprise additives, such as: (a) disintegrating agents; (b) dispersing agents; (c) wetting agents; (d) at least one preservative, (e) viscosity enhancing agents, (f) at least one sweetening agent, (g) at least one flavoring agent, (h) a complexing agent, and (i) an ionic dispersion modulator. In some embodiments, the aqueous dispersions can further comprise a crystalline inhibitor.

Examples of disintegrating agents for use in the aqueous suspensions and dispersions include, but are not limited to, a starch, e.g., a natural starch such as corn starch or potato starch, a pregelatinized starch such as National 1551 or Amijele®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol(®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 9080, also known as Poloxamine 9080, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908%).

Wetting agents (including surfactants) suitable for the aqueous suspensions and dispersions described herein are known in the art and include, but are not limited to, acetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450), and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben) and their salts, benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth. In one embodiment, the aqueous liquid dispersion can comprise methylparaben and propylparaben in a concentration ranging from about 0.01% to about 0.3% methylparaben by weight to the weight of the aqueous dispersion and 0.005% to 0.03% propylparaben by weight to the total aqueous dispersion weight. In yet another embodiment, the aqueous liquid dispersion can comprise methylparaben 0.05 to about 0.1 weight % and propylparaben from 0.01-0.02 weight % of the aqueous dispersion.

Suitable viscosity enhancing agents for the aqueous suspensions or dispersions described herein include, but are not limited to, methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity enhancing agent will depend upon the agent selected and the viscosity desired.

Examples of natural and artificial sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet®. Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.0001% to about 10.0% the weight of the aqueous dispersion. In another embodiment, the aqueous liquid dispersion can comprise a sweetening agent or flavoring agent in a concentration ranging from about 0.0005% to about 5.0% wt % of the aqueous dispersion. In yet another embodiment, the aqueous liquid dispersion can comprise a sweetening agent or flavoring agent in a concentration ranging from about 0.0001% to 0.1 wt %, from about 0.001% to about 0.01 weight %, or from 0.0005% to 0.004% of the aqueous dispersion.

In addition to the additives listed above, the liquid ganaxolone formulations can also comprise inert diluents commonly used in the art, such as water or other solvents, solubilizing agents, and emulsifiers.

In some embodiments, the ganaxolone formulations can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art include, but are not limited to, for example, U.S. Pat. Nos. 5,858,401, 6,667,048, and 6,960,563.

Exemplary emulsifiers are ethyl alcohol, isopropyl alcohol, ethyl carbonate, ethyl acetate, benzyl alcohol, benzyl benzoate, propylene glycol, 1,3-butyleneglycol, dimethylformamide, sodium lauryl sulfate, sodium docusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In certain preferred embodiments, the liquid pharmaceutical formulation comprises ganaxolone, hydroxypropyl methylcellulose, polyvinyl alcohol, sodium lauryl sulfate, simethicone, methyl paraben, propyl paraben, sodium benzoate, citric acid, and sodium citrate at pH 3.8-4.2. The suspension may comprise ganaxolone at a concentration of 50 mg/ml. The formulation may further comprise a pharmaceutically acceptable sweetener (e.g., sucralose) and/or a pharmaceutically acceptable flavorant (e.g., cherry). The formulation may be enclosed, e.g., in a 120 mL, 180 mL, 240 mL, or 480 mL bottle.

A formulation for oral administration may be an oral solid dosage form (e.g., an oral capsule or tablet) or a liquid (e.g., an oral suspension comprising ganaxolone). In certain embodiments, the oral suspension is administered to the patient via the use of an oral syringe.

In certain preferred embodiments, the oral solid formulation of the present invention may be a formulation as described and prepared in Applicant's prior U.S. Pat. No. 7,858,609, entitled “Solid Ganaxolone Formulations and Methods for the Making and Use Thereof”, hereby incorporated by reference in its entirety. However, the oral solid dosage formulation of ganaxolone may be prepared in accordance with other methods known to those skilled in the art.

For example, as disclosed in U.S. Pat. No. 7,858,609, the oral solid formulation may comprise stabilized particles comprising ganaxolone, a hydrophilic polymer, a wetting agent, and an effective amount of a complexing agent that stabilizes particle growth after an initial particle growth and endpoint is reached, the complexing agent being a small organic molecule having a molecular weight less than 550 and containing a moiety selected from the group consisting of a phenol moiety, an aromatic ester moiety and an aromatic acid moiety, wherein the stabilized particles have a volume weighted median diameter (D50) of the particles is from about 50 nm to about 500 nm, the complexing agent being present in an amount from about 0.05% to about 5% w/w, based on the weight particles of the solid. The hydrophilic polymer may be in an amount from about 3% to about 50%, w/w, based on the weight of the solid particles. The wetting agent may be an amount from about 0.01% to about 10%, w/w, based on the weight of the solid particles. Ganaxolone may be in an amount from about 10% to about 80% (and in certain embodiments form about 50% to about 80%) based on the weight of the stabilized particles. The stabilized particles may exhibit an increase in volume weighted median diameter (D50) of not more than about 150% when the particles are dispersed in simulated gastric fluid (SGF) or simulated intestinal fluid (SIF) at a concentration of 0.5 to 1 mg ganaxolone/mL and placed in a heated bath at 36° to 38° C. for 1 hour as compared to the D50 of the stabilized particles when the particles are dispersed in distilled water under the same conditions, wherein the volume weighted median diameter (D50) of the stabilized particles dispersed in SGF or SIF is less than about 750 nm. The stabilized particles may exhibit an increase in volume weighted median diameter (D50) of not more than about 150% when the formulation is dispersed in 15 mL of SGF or SIF at a concentration of 0.5 to 1 mg ganaxolone/mL as compared to the D50 of the stabilized particles when the particles are dispersed in distilled water under the same conditions, wherein the volume weighted median diameter (D50) of the stabilized particles dispersed in SGF or SIF is less than about 750 nm. The solid stabilized particles may be combined with optional excipients and prepared for administration in the form of a powder, or they may be incorporated into a dosage form selected from the group consisting of a tablet or capsule. The complexing agent may be a paraben, benzoic acid, phenol, sodium benzoate, methyl anthranilate, and the like. The hydrophilic polymer may be a cellulosic polymer, a vinyl polymer and mixtures thereof. The cellulosic polymer may be a cellulose ether, e.g., hydroxypropymethylcellulose. The vinyl polymer may be polyvinyl alcohol, e.g., vinyl pyrrolidone/vinyl acetate copolymer (S630). The wetting agent may be sodium lauryl sulfate, a pharmaceutically acceptable salt of docusate, and mixtures thereof. When the particles are incorporated into a solid dosage form, the solid dosage form may further comprise at least one pharmaceutically acceptable excipient, e.g., an ionic dispersion modulator, a water soluble spacer, a disintegrant, a binder, a surfactant, a plasticizer, a lubricant, a diluent and any combinations or mixtures thereof. The water soluble spacer may be a saccharide or an ammonium salt, e.g., fructose, sucrose, glucose, lactose, mannitol. The surfactant may be, e.g., polysorbate. The plasticizer may be, e.g., polyethylene glycol. The disintegrant may be cross-linked sodium carboxymethylcellulose, crospovidone, mixtures thereof, and the like.

A capsule may be prepared, e.g., by placing the bulk blend ganaxolone formulation, described herein, inside of a capsule. In some embodiments, the ganaxolone formulations (non-aqueous suspensions and solutions) are placed in a soft gelatin capsule. In other embodiments, the ganaxolone formulations are placed in standard gelatin capsules or non-gelatin capsules such as capsules comprising HPMC. In other embodiments, the ganaxolone formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the ganaxolone formulation is delivered in a capsule form.

Preferably, each capsule contains about 200 to about 600 mg ganaxolone, about 300 to about 600 mg ganaxolone, about 400 to about 600 mg ganaxolone, about 500 to about 600 mg ganaxolone, about 200 mg ganaxolone, about 250 mg ganaxolone, about 300 mg ganaxolone, about 500 mg ganaxolone or about 600 mg ganaxolone. In certain embodiments, each capsule contains either 200 mg or 225 mg ganaxolone, and hydroxypropyl methylcellulose, sucrose, polyethylene glycol 3350, polyethylene glycol 400, sodium lauryl sulfate, sodium benzoate, citric acid anhydrous, sodium methyl paraben, microcrystalline cellulose, 30% Simethicone Emulsion, gelatin capsules, polysorbate 80, and sodium chloride. In some of the embodiments, the size of the capsule is 00.

Alternatively, the oral dosage forms of the present invention may be in the form of a controlled release dosage form, as described in U.S. Pat. No. 7,858,609.

In certain preferred embodiments, the oral solid formulation of the present invention may be a formulation as described and prepared U.S. Pat. No. 8,367,651.

As described in U.S. Pat. No. 8,367,651, solid stabilized particles may comprise ganaxolone, a hydrophilic polymer, a wetting agent, and an effective amount of a complexing agent that stabilizes particle growth after an initial particle growth and endpoint is reached, the complexing agent being a small organic molecule having a molecular weight less than 550 and containing a moiety selected from the group consisting of a phenol moiety, an aromatic ester moiety and an aromatic acid moiety, wherein the stabilized particles have a volume weighted median diameter (D50) of the particles is from about 50 nm to about 500 nm and the concentration of ganaxolone in the solid stabilized particles is at least 50% by weight. The hydrophilic polymer maybe in an amount from about 3% to about 50%, w/w, based on the weight of the solid particles. The wetting agent may be in an amount from about 0.01% to about 10%, w/w, based on the weight of the solid particles. In some of the embodiments, the stabilized particles exhibit an increase in volume weighted median diameter (D50) of not more than about 150% when the particles are dispersed in simulated gastric fluid (SGF) or simulated intestinal fluid (SIF) at a concentration of 0.5 to 1 mg ganaxolone/mL and placed in a heated bath at 36° to 38° C. for 1 hour as compared to the D50 of the stabilized particles when the particles are dispersed in distilled water under the same conditions, wherein the volume weighted median diameter (D50) of the stabilized particles dispersed in SGF or SIF is less than about 750 nm. In some embodiments, the stabilized particles exhibit an increase in volume weighted median diameter (D50) of not more than about 150% when the formulation is dispersed in 15 mL of SGF or SIF at a concentration of 0.5 to 1 mg ganaxolone/mL as compared to the D50 of the stabilized particles when the particles are dispersed in distilled water under the same conditions, wherein the volume weighted median diameter (D50) of the stabilized particles dispersed in SGF or SIF is less than about 750 nm. In some embodiments, ganaxolone may be present in an amount greater than 50% to about 80%, based on the weight of the particles. In some embodiments, the stabilized particles may exhibit an increase in volume weighted median diameter (D50) of not more than about 150% when the particles are dispersed in simulated gastric fluid (SGF) or simulated intestinal fluid (SIF) at a concentration of 0.5 to 1 mg ganaxolone/mL and placed in a heated bath at 36° to 38° C. for 1 hour, as compared to the D50 of the stabilized particles when the particles are dispersed in distilled water under the same conditions, wherein the volume weighted median diameter (D50) of the stabilized particles dispersed in SGF or SIF is less than about 750 nm. In some embodiments, the solid stabilized particles may be in the form of a powder. In some embodiments, the particles may be incorporated into a dosage form selected from the group consisting of a tablet or capsule. In some embodiments, the volume weighted median diameter (D50) of the stabilized particles dispersed in distilled water is from about 100 nm to about 350 nm. In some embodiments, the complexing agent is selected from the group consisting of parabens, benzoic acid, methyl anthranilate, and pharmaceutically acceptable salts thereof and mixtures thereof. In some embodiments, paraben is selected from the group consisting of methylparaben, ethylparaben, propylparaben, pharmaceutically acceptable salts thereof and mixtures thereof. In some embodiments, the hydrophilic polymer is selected from the group consisting of a cellulosic polymer, a vinyl polymer and mixtures thereof. In some embodiments, the cellulosic polymer is a cellulose ether. In some embodiments, the cellulose ether is hydroxypropylmethylcellulose. In some embodiments, the vinyl polymer is polyvinyl alcohol. In some embodiments, the wetting agent is selected from the group consisting of sodium lauryl sulfate, a pharmaceutically acceptable salt of docusate, and mixtures thereof. Is some embodiments, the particles are incorporated into a solid dosage form, further comprising at least one pharmaceutically acceptable excipient selected from the group consisting of an ionic dispersion modulator, an water soluble spacer, a disintegrant, a binder, a surfactant, a plasticizer, a lubricant, and any combinations or mixtures thereof. In some embodiments, the pharmaceutically acceptable excipient comprises an ionic dispersion modulator. In some embodiments, the ionic dispersion modulator is in an amount from about 1% to about 50%, w/w, based on the weight of the solid particles. In some embodiments, the ionic dispersion modulator is a salt. In some embodiments, the ionic dispersion modulator is an inorganic salt is selected from the group consisting of a magnesium salt, a calcium salt, a lithium salt, a potassium salt, a sodium salt and mixtures thereof. In some embodiments, the ionic dispersion modulator is an organic salt is selected from the group consisting of a citrate salt, a succinate salt, a fumarate salt, a malate salt, maleate salt, a tartrate salt, a glutarate salt, a lactate salt and mixtures thereof. In some embodiments, the pharmaceutically acceptable excipient comprises a water soluble spacer. In some embodiments, the water soluble spacer is in an amount from about 2% to about 60%, w/w, based on the weight of the solid particles. In some embodiments, the water soluble spacer is a saccharide or an ammonium salt. In some embodiments, the saccharide is selected from the group consisting of fructose, sucrose, glucose, lactose, mannitol and mixtures thereof. In some embodiments, the disintegrant is selected from the group consisting of cross-linked sodium carboxymethylcellulose, crospovidone and any combinations or mixtures thereof. In some embodiments, the surfactant is a polysorbate. In some embodiments, the plasticizer is polyethylene glycol. In some embodiments, the solid dosage form is an immediate release dosage form. In some embodiments, the solid dosage form is a controlled release dosage form. In some embodiments, the particles are incorporated into an oral solid dosage form comprising (i) a controlled release component comprising a first portion of the stabilized particles; and a controlled release material, and (ii) an immediate release component comprising a second portion of the stabilized particles, the first and second portion of stabilized particles having a volume weighted median diameter (D50) of from about 50 nm to about 500 nm. In some embodiments, the ratio of ganaxolone in controlled release to immediate release is from about 4:1 to about 1:4. In some embodiments, the dosage form provides a therapeutic effect for about 8 to about 24 hours after administration. In some embodiments, the complexing agent is in an amount from about 0.05% to about 5%, w/w, based on the weight of the solid particles. In some embodiments, the complexing agent comprises methylparaben or a salt thereof. In some embodiments, the complexing agent comprises benzoic acid or a salt thereof. In some embodiments, the complexing agent comprises methyl anthranilate. In some embodiments, the formulation includes from about 200 mg to about 800 mg ganaxolone.

As further described in U.S. Pat. No. 8,367,651, solid stabilized particles may also comprise ganaxolone, a hydrophilic polymer, a wetting agent, and an effective amount of a complexing agent selected from the group of small organic molecules having a molecular weight less than 550 and containing a moiety selected from the group consisting of a phenol moiety, an aromatic ester moiety and an aromatic acid moiety, the stabilized particles having a volume weighted median diameter (D50) of the particles from about 50 nm to about 500 nm, the concentration of ganaxolone in the solid stabilized particles being at least 50% by weight. In some embodiments, ganaxolone is present in an amount greater than 50% to about 80%, based on the weight of the particles. In some embodiments, the particles are incorporated into a dosage form selected from the group consisting of a tablet or capsule. In some embodiments, the complexing agent is selected from the group consisting of parabens, benzoic acid, methyl anthranilate, and pharmaceutically acceptable salts thereof and mixtures thereof.

In certain preferred embodiments, the formulation of the present invention may be a pharmaceutical composition described in U.S. Pat. No. 9,029,355.

In certain embodiments, the composition may comprise the ganaxolone nanoparticles as described above, further in formulations as described in U.S. Pat. No. 9,029,355. In some embodiments, the pharmaceutical composition is a compressed tablet. In some embodiments, the pharmaceutical composition is contained inside a capsule.

The formulation can be an intravenous formulation of ganaxolone. The intravenous formulation of ganaxolone can comprise a cyclodextrin (e.g., a sulfobutyl ether β-cyclodextrin (Captisol®). The IV solution can comprise a sterile ready to administer solution containing 1 mg/ml ganaxolone in Captisol® (Captisol®:GNX ratio 60:1). The ready to administer solution can comprise 1 mg/ml ganaxolone in sulfobutyl ether β-cyclodextrin (Captisol®) having a Captisol to ganaxolone ratio of 60:1, and a buffer (i.e., phosphate and/or sodium chloride). In embodiments, the IV solution is a sterile solution containing 3 mg/ml ganaxolone in Captisol® (Sulfobutylether-β-Cyclodextrin) (Captisol®: GNX ratio 70:1) or 5 mg/ml ganaxolone in Captisol, each of which may or may not be may be diluted with 0.9% saline (i.e., sodium chloride) solution, for example to produce a 1 mg/ml ganaxolone solution for administration, prior to administration.

In certain embodiments, the formulation (e.g., an intravenous formulation) comprises ganaxolone and sulfobutylether-β-cyclodextrin (e.g., Captisol®) in a weight ratio from about 1:50 to about 1:75. In some of these embodiments, the weight ratio ganaxolone and Captisol® is about 1:51, about 1:52, about 1:53, about 54:1, about 1:55, about 1:56, about 1:57, about 1:58, about 1:59, about 1:60, about 1:61, about 1:62, about 1:63, about 1:64, about 1:65, about 1:66, about 1:67, about 1:68, about 1:69, about 1:70, about 1:71, or about 1:72. In some of these embodiments, the weight ratio ganaxolone and Captisol® is about 1:60.

The intravenous formulation may be selected, e.g., from the group consisting of nanocrystal formulations; emulsions; lyocells; solvents or surfactants; liposomes; microemulsions; and liquids containing solid-lipid nanoparticles.

In certain embodiments, the intravenous formulation is an IV solution. An intravenous formulation is preferably a sterile liquid (e.g., aqueous liquid in the form of an emulsion, a suspension, a solution and the likes). In some of these embodiments, the IV solution comprises ganaxolone and a pharmaceutically acceptable solvent(s) and/or oil(s) that can solubilize ganaxolone.

In certain embodiments, the intravenous formulation is an oil-in-water emulsion.

In certain embodiments, the intravenous formulation is a liquid nanoparticulate formulation (e.g., a liquid comprising nanoparticles of ganaxolone). In some of the embodiments, the nanoparticulate formulation comprises ganaxolone and a polymeric and/or ionic stabilizer, and is free from complexing agents. In certain embodiments, the polymeric and ionic stabilizers are selected from the group consisting of surfactants. In certain embodiments, surfactants are selected from the group consisting of sorbitan esters, polyoxyethylene sorbitan fatty acid esters, poloxamers, cholesterol salts, and bile salts.

In certain embodiments, the formulation for the intravenous infusion may be a formulation as described and prepared in U.S. Patent Publication No. 2017/0258812 or U.S. Patent Publication No. 2016/0228454. However, formulations for the intravenous infusion may be prepared in accordance with other methods known to those skilled in the art.

As described in U.S. Patent Publication No. 2016/0228454, an aqueous injectable ganaxolone formulation may comprise a) ganaxolone and sulfobutyl ether-β-cyclodextrin in an inclusion complex; and b) water. In some embodiments, the complex comprising ganaxolone and sulfobutyl ether-5-cyclodextrin comprises a 1:1 ganaxolone: sulfobutyl ether-β-cyclodextrin complex; and the w/w ratio of sulfobutyl ether-β-cyclodextrin to ganaxolone is about 52:1 or greater. In some embodiments, the formulation may further comprise surfactant. In some embodiments, the surfactant is a sorbitan ester, a polyoxyethylene sorbitan fatty acid ester, a poloxamer, a cholesterol salt, or a bile salt. In some embodiments, the surfactant may comprise from about 1 to about 15 percent of the formulation by weight. In some embodiments, the surfactant is polysorbate 80. In some embodiments, the formulation further comprises a buffer and has a pH of about 6.0 to about 7.6. In some embodiments, the buffer is a phosphate buffer. In some embodiments, the buffer is a combination of a monobasic phosphate buffer and a dibasic phosphate buffer, wherein the concentration of each phosphate buffer is 2 mM to 50 mM. In some embodiments, the buffer is a phosphate buffer. In some embodiments, the buffer is a combination of a monobasic phosphate buffer and a dibasic phosphate buffer, wherein the concentration of each phosphate buffer is 2 mM to 50 mM. In some embodiments, the concentration of ganaxolone is 2 mg/ml to 8 mg/ml, the w/w ratio of sulfobutyl ether-β-cyclodextrin to ganaxolone is within the range from about 52:1 to about 90:1; the formulation contains a buffer and has a pH of 6.7 to 7.3 or a pH of 6.0 to 7.0; and the formulation contains from 1 to 15 weight percent surfactant. In some embodiments, the concentration of ganaxolone is 1 mg/ml to 5 mg/ml; the weight percent of sulfobutyl ether-β-cyclodextrin 25% to 35%; and the formulation contains from 5% to 15% (weight percent) of at least one of the following: a surfactant, ethanol, glycerin, or propylene glycol. In some embodiments, the formulation further comprises a preservative. In some embodiments, the preservative is benzyl alcohol, chlorbutanol, 2-ethoxyethanol, parabens (including methyl, ethyl, propyl, butyl, and combinations), benzoic acid, sorbic acid, chlorhexidene, phenol, 3-cresol, thimerosal, or a phenylmercurate salt.

As further described in U.S. Patent Publication No. 2016/0228454, the formulation may be a lyophilized ganaxolone formulation comprising ganaxolone and sulfobutyl ether-O-cyclodextrin, wherein the ganaxolone formulation is 1.0% to 1.5% ganaxolone. In some embodiments, the formulation may further comprise a bulking agent. In some embodiments, the bulking agent is mannitol, lactose, sucrose, trehalose, sorbitol, glucose, rafinose, glycine, histidine, polyethylene glycol (PEG), or polyvinyl pyrrolidone (PVP).

Ganaxolone formulations suitable for parenteral administration in the methods of the present invention may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, ganaxolone can be dissolved at concentrations of >1 mg/ml using water soluble beta cyclodextrins (e.g. beta-sulfobutyl-cyclodextrin and 2-hydroxypropylbetacyclodextrin). A particularly suitable cyclodextrin is a substituted-β-cyclodextrin is Captisol®. Proper fluidity can be maintained, for example, by the use of a coating such as lecithin, by the maintenance of the required particle size in the case of dispersions, and by the use of surfactants. Ganaxolone formulations suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. Ganaxolone suspension formulations designed for extended release via subcutaneous or intramuscular injection can avoid first pass metabolism and lower dosages of ganaxolone will be necessary to maintain plasma levels of about 50 ng/ml. In such formulations, the particle size of the ganaxolone particles and the range of the particle sizes of the ganaxolone particles can be used to control the release of the drug by controlling the rate of dissolution in fat or muscle.

In certain embodiments, the intravenous formulation is a solution comprising a complexing agent(s). In some of these embodiments, a complexing agent is a molecule with a lipophilic core and hydrophilic outer shell capable of solubilizing ganaxolone

In certain embodiments, the formulation is an IV solution comprising ganaxolone and sulfobutylether cyclodextrin (Captisol®), wherein ganaxolone is solubilized in sulfobutylether cyclodextrin (Captisol®). In some embodiments, the solution comprises 3 mg of ganaxolone per 1 ml of the solution and is sterile. In certain embodiments, the solution is stable for at least 18 months, is stored refrigerated at a temperature from about 4° C. to about 8° C.

### C. Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

The abbreviation “EEG” means electroencephalography.

The terms “subject” and “patient” are used interchangeably herein to refer to any animal, such as any mamma, including but not limited to, humans, non-human primates, rodents, and the like. The mammal is preferably a human.

The term “effective amount” or “therapeutically effective amount” as used herein refers to an amount of a compound described herein (e.g., ganaxolone) that is sufficient to effect the intended result, including, but not limited to disease treatment as illustrated below. The “therapeutically effective amount” can be an amount effective to manage seizure activity, suppress seizure, allow the patient to recover from a hyperexcitable state, prevents seizure-relapse, or can provide continued suppression of seizure. The therapeutically effective amount can vary depending upon the intended application, or the subject and the disease condition being treated, e.g., the weight and age of the subject, the severity of the disease condition, the manner of administration and the like.

The term “pharmaceutical compositions” as used herein are compositions comprising at least one active agent, such as a compound or salt, solvate, or hydrate of ganaxolone, and at least one other substance, such as a carrier. Pharmaceutical compositions optionally contain one or more additional active agents. When specified, pharmaceutical compositions meet the U.S. FDA's GMP (good manufacturing practice) standards for human or non-human drugs. “Pharmaceutical combinations” are combinations of at least two active agents which may be combined in a single dosage form or provided together in separate dosage forms with instructions that the active agents are to be used together to treat a disorder, such as status epilepticus.

As used herein, the terms “treat,” treatment,” or“treating” and grammatically related terms, refer to an improvement of any sign, symptoms, or consequence of the disease, such as prolonged survival, less morbidity, and/or a lessening of side effects.

### 3. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

### 4. EXAMPLES

### Example 1: Preliminary Pharmacokinetic and Pharmacodynamic (PKIPD) Analysis

A preliminary PK/PD analysis was conducted to explore the relationship, if any, between ganaxolone levels and percent change in major motor seizure frequency.

a) Study Design

Global, randomized, double-blind, placebo-controlled phase 3 clinical trial to assess the safety and efficacy of adjunctive ganaxolone for the treatment of seizures associated with CDD. Patients aged 2 to 21 years with a pathogenic or likely pathogenic mutation of the CDKL5 gene, neurodevelopmental impairment, and seizures refractory to treatment with at least 2 prior antiseizure medications who experienced at least 16 seizures per 28 days during the 2 months prior to screening were eligible to enroll. Study consisted of a 6-week baseline followed by a 17-week double-blind phase (ganaxolone or placebo, 1:1). The dose of ganaxolone 50 mg/mL suspension was titrated over 4 weeks to 63 mg/kg/day (21 mg/kg TID), not to exceed 1800 mg/d (600 mg TID), or to the maximum tolerated dose. Blood draws for PK analysis were scheduled to occur at visit 3 (week 5), visit 4 (week 9), and visit 5 (week 17).

b) Methods

Mean ganaxolone concentration was calculated for each subject using available results from up to three laboratory determinations during the double-blind phase. Linear regression was conducted using arithmetic- and natural logarithm-transformed percent reduction in major motor seizures (logₑ [percent reduction+100]) as dependent variable and natural logarithm-transformed mean ganaxolone concentration as the single explanatory variable. Regression diagnostics included examination of residual and normal probability plots and determination of outliers and influential values. Cases with standardized residuals >2 or <−2 were excluded from the model and the regression was repeated.

The resulting sample was utilized in determination of a Pearson correlation coefficient (using log-transformed values). Additionally, percent seizure reduction was compared in three tertiles representing low (N=13), medium (N=13) and high (N=12) mean per subject ganaxolone concentrations using a Kruskal-Wallis test.

The number of CNS-related adverse events suggestive of potential dose-related toxicity (somnolence, sedation, lethargy, disturbance in attention, drooling and hypotonia) in ganaxolone-treated participants as well as the onset and duration of the events was tabulated, and the number of participants with CNS adverse events during each week of the double-blind phase calculated.

c) Resubs

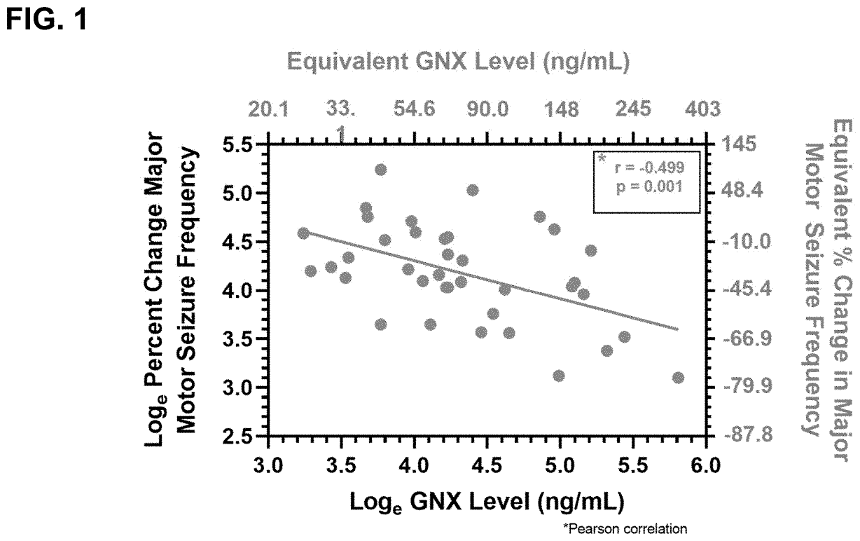

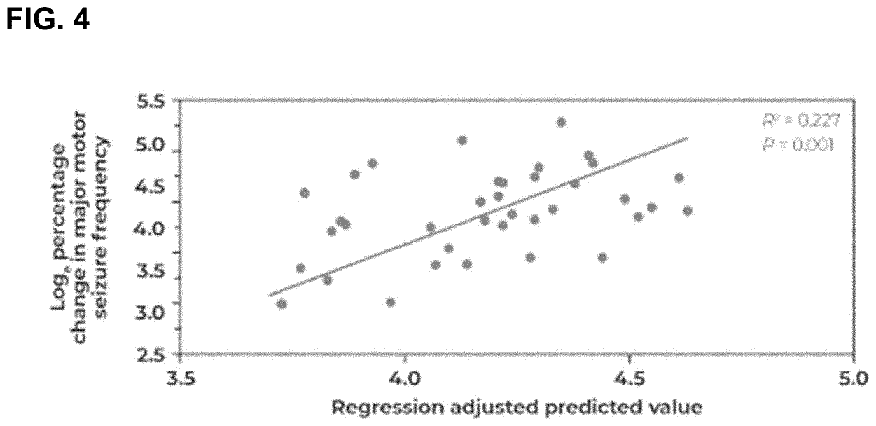

Forty-four participants with seizure reduction data had at least one plasma ganaxolone level determination (mean+standard deviation=103.5+79.2 ng/mL). In a linear regression with percent seizure reduction as the dependent variable and mean plasma ganaxolone concentration as the independent variable, six cases were determined to be outliers due to adjusted residuals >2 or <−2. Repeat linear regression excluding those cases (N=38) yielded an adjusted R2 of 0.227 (F(1,36)=11.89), p=0.001). (FIG. 4) The correlation coefficient for mean plasma ganaxolone concentration and percent reduction in major motor seizures using the same sample was −0.499 (p=0.001) (FIG. 1). A robust regression was performed including all observations (N=44) which replicated the findings of this analysis.

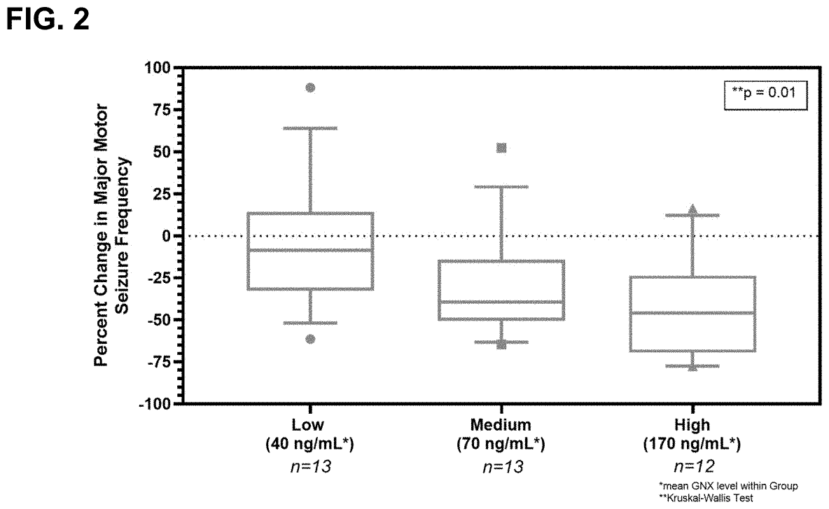

Mean and median percent reduction in major motor seizures was calculated for low-, medium- and high-ganaxolone concentration tertiles (Table 1). There was a statistically significant between-group difference in the percent reduction of major motor seizure frequency (H(2)=9.087, p=0.011)(FIG. 2). Post-hoc pairwise comparisons of sample distributions for the three groups showed a statistically significant difference between Low and High GNX level groups but not for other between-group tests.

TABLE 1
Tertiles Based on Mean Ganaxolone Plasma Concentration

| | | Mean percentage | Median percentage |
|---|---|---|---|
| | Mean GNX | change in major | change in major |
| GNX level | level | motor seizures | motor seizures |
| groups | (ng/ml) | (per 28 days) | (per 28 days) |
| Low (n = 13) | 40.2 | −6.5 | −8.4 |
| Median (n = 13) | 72.3 | −30.3 | −39.5 |
| High (n = 12) | 172.6 | −44.3 | −46.0 |

In summary, logarithms of plasma ganaxolone levels and percent change in major motor seizure frequency were negatively correlated. Increases in plasma GNX levels were associated with greater reductions in seizure frequency in the range of 27 to 333 ng/mL in patients with CDKL5 deficiency disorder (CDD). The back-transformation of log values indicates that a plasma concentration of approximately 100 ng/mL (mean of CDD population) predicts an approximately 40% seizure reduction in the participants in this study.

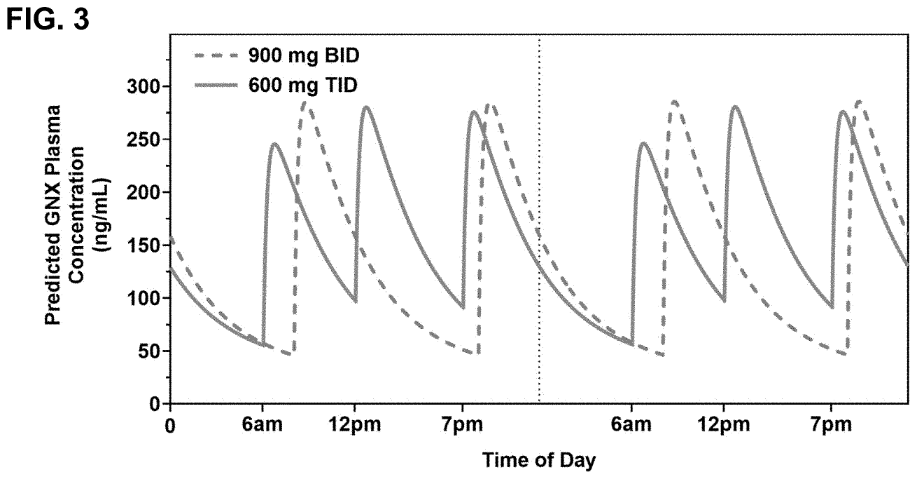

Modeled PK curves based on previous Phase 1 studies demonstrate that TID dosing is able to increase trough GNX levels compared to BID dosing (FIG. 3). Results from the preliminary PK/PD analysis suggest that increasing plasma ganaxolone concentrations were associated with improved seizure reduction and that concentrations of approximately 100 ng/mL are associated with meaningful changes in seizure frequency. Based on the modeled PK curves, TID dosing may yield plasma ganaxolone levels >100 ng/mL for approximately 78% of a 24-hr day compared to just 53% using BID dosing. While these analyses do not preserve randomization, and hence might not represent causal effects of GNX on changes in seizure frequency, they are suggestive that TID dosing might provide increased antiseizure benefits.

### Example 2: Study in Adults with Focal Seizures

A double-blind, placebo-controlled study that enrolled 52 participants 18-65 years of age undergoing AED discontinuation during inpatient continuous EEG monitoring as part of an evaluation for surgical treatment of epilepsy was performed. Participants were randomized and administered: (i) ganaxolone 500 mg TID, (ii) 625 mg TID, or (iii) matching placebo and treated for up to 8 days. The primary endpoint was difference in the duration of treatment before meeting withdrawal criteria based on seizure occurrence. A log-rank test demonstrated a longer duration of treatment prior to study withdrawal for ganaxolone relative to placebo, the result of which approached statistical significance (p=0.08).

A Phase 2 study was conducted. It enrolled 147 participants 18-69 years of age with a diagnosis of epilepsy with uncontrolled partial-onset (focal) seizures with or without secondary generalization despite being treated with up to 3 AEDs. Participants were randomized to adjunctive placebo (N=49) or GNX liquid suspension (N=98) titrated to a target dose of at 500 mg TID (1500 mg/day) in addition to their baseline AEDs, after which they entered an eight-week prospective baseline period followed by a 10-week double-blind phase. The primary endpoint was the reduction in weekly frequency of focal motor, focal impaired awareness and focal to bilateral tonic-clonic seizures from baseline to the double-blind phase. The prespecified primary analysis was the reduction in mean weekly seizure frequency, with log-transformed rates analyzed using analysis of covariance (ANCOVA). The protocol defined the population for analysis (“Primary Population”) as participants with 10 or fewer seizures/week during the baseline period. The weekly seizure frequency in the GNX group decreased from 3.13 during the baseline period to 2.70 during the double-blind phase, with an increase in the placebo group from 2.84 to 3.08, a net reduction of 11.8% for GNX compared to placebo (p=0.042). In the ITT population, defined as randomized participants who received at least one dose of study medication, the net reduction was 11.4% (p=0.049). The median percent reduction in the ITT population was 25.9% in the ganaxolone group vs. 10.2% for placebo (p=0.014).

Based on the results of the phase 2 study, a double-blind, placebo-controlled phase 3 trial in adults with focal seizures was conducted. The study enrolled two cohorts: Cohort 1 was an initial assessment of safety and tolerability of GNX 1200 or 1800 mg/day compared to placebo. Cohort 2, on which the primary efficacy analysis was based, enrolled 359 participants with a diagnosis of epilepsy with uncontrolled partial-onset (focal) seizures with or without secondary generalization who had failed at least two prior AEDs and had been on a stable regimen of one to three AEDs for at least one month. Following an eight-week prospective baseline period during which there were at least three seizures/28 days, participants were randomized to GNX 1800 mg/d (N=179) or placebo (N=180). The primary endpoint was the percent change in 28-day frequency of focal motor, focal impaired awareness and focal to bilateral tonic-clonic seizures from baseline to the double-blind phase. The primary efficacy analysis was a rank ANCOVA based on the modified intent-to-treat (mITT) population, defined as randomized participants who had taken at least one dose of study drug and provided post-baseline seizure data. The percent reduction in seizure frequency from the baseline period to the double-blind phase was 21.3% compared to 10.3 for ganaxolone and placebo, respectively (p=0.179).

An open label extension was performed in which participants received ganaxolone at doses from 900-1800 mg daily in divided doses.

Despite comparable patient populations, study endpoints and daily doses of ganaxolone (1500 mg/day in Phase 2 and 1800 mg in Phase 3), the Phase 3 failed to replicate the efficacy demonstrated in the Phase 2 study (Table 1).

TABLE 2
Comparison of the Phase 2 study and the Phase 3 study.

| | | | | Median % | Median % | |
|---|---|---|---|---|---|---|
| | N, | | | reduction | reduction | P- |
| Phase | GNX/PBO | Dose | Schedule | GNX | PBO | value |
| 2 | 98/49 | 1500 mg/d | TID | 25.9% | 10.2% | 0.014 |
| 3 | 179/180 | 1800 mg/d | BID | 21.3% | 10.3% | 0.179 |

Notably, the dosing schedule was changed from three times daily in Phase 2 to twice daily in Phase 3. Based on the pharmacokinetics of ganaxolone and the modeled plasma concentration data illustrated in FIG. 3, a likely explanation for the difference in study outcomes in the greater peak-trough variability seen with BID compared to TID dosing.

1. A method for treating seizure or an epilepsy disorder comprising administering to a subject in need thereof ganaxolone in a therapeutically effective amount that produces a ganaxolone plasma concentration of at least about 100 ng/ml or greater for approximately about 70% or more of a 24 day.

2. The method of claim 1, wherein ganaxolone is administered three times per day.

3. The method of claim 1, wherein ganaxolone is administered orally.

4. The method of claim 3, wherein ganaxolone is administered as an oral suspension.

5. The method of claim 3, wherein ganaxolone is administered as an oral capsule.

6. The method of claim 1, wherein ganaxolone is administered in an amount of up to 63 mg/kg/day.

7. The method of claim 1, wherein ganaxolone is administered in an amount of up to 1,800 mg per day.

8. The method of claim 1, wherein ganaxolone is administered in an amount of up to 1,500 mg per day.

9. The method of claim 1, wherein the epilepsy disorder is a focal seizure, a generalized seizure, progressive myoclonic epilepsy, reflex epilepsy, Landau-Kleffner Syndrome, Ohtahara syndrome, Rasmussen's syndrome, infantile spasms (or West syndrome), Lennox-Gastaut syndrome (LGS), Rett syndrome, Dravet syndrome, Doose syndrome, CDKL5 disorder, intractable childhood epilepsy (ICE), childhood absence epilepsy (CAE), juvenile myoclonic epilepsy (JME), essential tremor, acute repetitive seizures, benign rolandic epilepsy, status epilepticus, refractory status epilepticus, super-refractory status epilepticus, PCDH19 pediatric epilepsy, increased seizure activity, a breakthrough seizures or an infantile spasms.

10. The method of claim 1, wherein administering ganaxolone reduces the frequency of seizure in the subject relative to baseline.

11. The method of claim 1, wherein administering ganaxolone reduces major motor frequency in the subject relative to baseline.

12. The method of claim 1, wherein administering ganaxolone results in a reduction in seizure frequency of about 20% or greater relative to baseline seizure frequency.

13. The method of claim 1, wherein administering ganaxolone results in a reduction in seizure frequency of at least about 35% or greater relative to baseline seizure frequency.

14. The method of claim 1, wherein the subject is monitored by electroencephalogram (EEG).

15. The method of claim 1, wherein seizure activity in the subject is monitored by electroencephalogram (EEG).

16. The method of claim 1, wherein ganaxolone or a pharmaceutically acceptable salt thereof is administered in an amount sufficient to produces a ganaxolone plasma concentration of at least about 100 ng/ml or greater for approximately 75% or more of a 24 hour day.

17. The method of claim 1, wherein 500 mg of ganaxolone is administered to the subject three times per day.

18. The method of claim 1, wherein 600 mg of ganaxolone is administered three times per day.

19. The method of claim 1, wherein about 200 mg to about 600 mg, about 300 mg to about 600 mg, about 400 mg to about 600 mg or about 500 mg to about 600 mg of ganaxolone is administered orally to the subject three times a day.
